(19) United States
(12) Patent Application Publication
(10) Pub. No.: US 2011/0318279 A1
(43) Pub. Date: Dec. 29, 2011

(54) POLYMERIC MASTICATORY MASSES FOR COSMETIC PRODUCTS

(75) Inventors: Meike Niesten, Koln (DE); Steffen Hofacker, Odenthal (DE); Thorsten Rische, Unna (DE); Sebastian Dörr, Dusseldorf (DE); Thomas Feller, Solingen (DE); Thomas Michaelis, Leverkusen (DE)

(73) Assignee: BAYERMATERIALSECIENCE AG, LEVERKUSEN (DE)

(21) Appl. No.: 13/127,677

(22) PCT Filed: Apr. 12, 2007
(86) PCT No.: PCT/EP07/03244

§ 371 Date: Sep. 14, 2011

(30) Foreign Application Priority Data
Apr. 22, 2006 (DE) 10-2006-018-826.8
Apr. 26, 2006 (DE) 10-2006-019-742.9

(51) Int. Cl. A61K 8/04; C08G 18/06; C08G 77/04; C08F 110/02; C08F 218/08; C08F 36/08; A61Q 11/00; C08J 9/30

(52) U.S. Cl. 424/48; 521/170; 521/73; 521/143; 521/149; 521/150; 521/154

(57) ABSTRACT

The invention relates to novel masticatory masses for oral hygiene, made from foamed synthetic polymers, a method for production and use thereof.

The invention relates to novel gum bases for the oral care sector which are based on foamed synthetic polymers, a method for their production and also use thereof.

Organic polymers are of wide occurrence as raw materials in cosmetic products. They may be found in many cosmetic products such as, for example, hair sprays, hair gels, mascara, lipsticks, creams, etc. In the oral care sector, polymers may be found, for example, in the form of toothbrushes, dental flosses, etc.

Owing to the developing requirement of society for oral care for the period between meals or after consumption, for example, of a between-meal snack (such as for example, sweets, nicotine, alcohol, etc.) or else on account of increased mobility (for example during air or train travel) in which conventional teeth cleaning with water, toothpaste and toothbrush is not possible, in the past products such as dental care chewing gums or else dental care wipes have been developed.

Dental care chewing gums essentially consist of gum base. This in turn consists of natural or synthetic polymers such as, for example, latex, polyvinyl ethers, polyisobutylene vinyl ethers, polyisobutene, etc. Such dental care chewing gums, as dental care compositions, generally contain pH-controlling substances which thus counteract the development of tooth decay (caries). Owing to their plastic behaviour, such dental care chewing gums, however, scarcely contribute to cleaning the chewing surfaces or tooth sides. In addition, chewing gums generally have the disadvantage that they must frequently be mechanically removed from public streets and spaces, and disposed of, which leads to considerable cleaning expenditure, on account of their adhesive properties, of floor and road surfaces.

Teeth wipes (for example Oral-B Brush Aways™, Gillette GmbH & Co. OHG, Germany) are distinguished in that they achieve good cleaning action of the tooth sides by applying the teeth wipe onto a finger and by rubbing the teeth. However, the mode of employing such teeth cleaning wipes in public has gained little acceptance for aesthetic reasons and is thus not an alternative to using a conventional toothbrush.

It has now been found that foamed materials may be produced from synthetic or chemically modified natural polymers, which foamed materials, inter alia owing to their particularly advantageous mechanical properties, are suitable as gum bases for the oral care sector.

The invention therefore relates to gum bases made from synthetic or natural chemically modified polymers.

A property of the gum bases which is essential to the invention is that they exhibit shape stability during chewing, that is to say do not undergo plastic deformation, as do, for example, chewing gums of the prior art, but rather, after stretching in a chewing process, return to their original shape owing to the polymer restoration forces present. This first ensures that a tooth-cleaning action (especially also e tooth sides) can also occur.

It is preferable when the gum bases of the invention have a tensile modulus at 100% extension of 0.1 to 8.0 MPa, at a tensile strength of 0.5 to 80 MPa and an extensibility of 100 to 3000%.

Particular preference is given to those which have a tensile modulus at 100% extension of 0.3 to 3.5 MPa, at a tensile strength of 0.5 to 40 MPa and an extensibility of 200 to 2000%.

The extension tests were carried out as specified in DIN 53504 using a dumbbell-shaped S2 sample body as specified in DIN 53504. The test moduli were determined as specified in DIN EN ISO 527. The layer thickness of the sample body was 2.5 mm±1 mm.

In addition, it is advantageous when the ratio of tensile strength and modulus of elasticity of the polymeric gum base according to the invention is greater than or equal to 1, preferably greater than 1.5, and particularly preferably greater than 2, and the ratio of the product of resistance to tear (as specified in DIN ISO 34-1 (2004)) and modulus of elasticity to the square of the tensile strength is less than 4 mm, preferably less than 1.5 mm.

In addition, the stability of the polymeric gum base under compression should be greater than 50 MPa, preferably greater than 75 MPa.

The present invention further relates to a method for producing the gum bases of the invention in which the synthetic or chemically modified natural polymers or the starting materials necessary for their formation, if appropriate together with further components of the gum bases, are foamed and simultaneously or subsequently cured to obtain the foam structure.

As synthetic polymers, in principle, all synthetic or chemically modified natural polymeric materials which are known as such to those skilled in the art come into consideration which may be foamed optionally with the aid of propellant gases or mechanical energy. It can be advantageous in this case when foam aids are added in order to obtain a stable foam structure.

Such foamable synthetic polymers can be polyurethane soft foams obtainable from one or more (poly)isocyanates and one or more polyol components, or else based on thermoplastic polyurethanes or based on aqueous polyurethane dispersions.

Those which are likewise suitable are, for example, polyvinyl chloride plastisols, low density polyethylene (LDPE), ethylene vinyl acetate-copolymers (EVA), synthetic or natural rubbers, silicone rubbers and also mixtures thereof.

Fundamentals for producing foams are described, for example, in “Fundamentals of Foam Formation” (J. H. Saunders, Chapter 2, Polymer Foams, Editors Klempner, Frisch Carl Hamer Verlag Munich, 1991)

In order to be able to foam the synthetic polymers according to the invention, they are preferably first prepared as liquid phase. If the components of the foams are not present as liquid per se, this can be performed by dissolving non-liquid components in a liquid component. For this the use of organic solvents, plasticisers, water or melting is likewise possible in order to provide the components in a phase liquid under foaming conditions, for example as solution, dispersion or melt.

The actual foaming proceeds by introducing air, nitrogen gas, low-boiling liquids such as pentane, chlorofluorocarbons, methylene chloride or else by chemical reactions such as the release of CO₂ by chemical reaction of isocyanate with water.

Curing with the foam structure being obtained can be initiated already during the step of foaming. This is, for example, the case when isocyanate/polyol mixtures are used for forming the synthetic polymer.

Curing subsequent to the foam formation proceeds, for example, with the use of aqueous polyurethane dispersions which are first foamed and not dried until thereafter.

Curing, in addition to chemical crosslinking or physical drying, can also proceed via temperature reduction of a melt, gellation of plastisols or coagulation, for example of lattices.

“Curing with the foam structure being obtained”, in the context of the present invention, means that the foamed mixture is converted into the solid state in such a manner that collapse of the foam with loss of the cell structure of the foam does not occur. In this case, then foams are obtained which, in a preferred embodiment, have the foam densities mentioned hereinafter.

Curing by physical drying preferably proceeds at a temperature of from 25 to 150° C., preferably 30° C. to 120° C., particularly preferably at 40 to 100° C. The drying can proceed in a conventional dryer.

In the production of the gum bases according to the invention, in addition to synthetic or chemically modified natural polymers or the starting materials necessary for their formation (I), use can also be made in conjunction of foam aids (II), crosslinkers (III), thickeners (IV), aids (V) and cosmetic additives (VI).

Suitable foam aids (II) are commercially conventional foam generators and/or stabilizers such as water-soluble fatty acid amides, sulphosuccinimides, hydrocarbon sulphonates, sulphates or fatty acid salts, the lipophilic radical preferably containing 12 to 24 carbon atoms.

Preferred foam aids (II) are alkanesulphonates or sulphates having 12 to 22 carbon atoms in the hydrocarbon radical, alkylbenzenesulphonates or sulphates having 14 to 24 carbon atoms in the hydrocarbon radical or fatty acid amides or fatty acid salts having 12 to 24 carbon atoms.

The abovementioned fatty acid amides are preferably fatty amides of mono- or di-(C2-C3-alkanol)amines. Fatty acid salts can be, for example, alkali metal salts, amine salts or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coconut fatty acid, tallow fatty acid, soya fatty acid and hydrogenation products thereof.

Particularly preferred foam aids (II) are sodium lauryl sulphate, sulphosuccinamides and ammonium stearates, and also mixtures thereof.

Suitable crosslinkers (III) are, for example, unblocked polyisocyanate crosslinkers, amide- and amine-formaldehyde resins, phenol resins, aldehyde and ketone resins, such as, for example, phenol-formaldehyde resins, resoles, furan resins, urea resins, carbamic ester resins, triazine resins, melamine resins, benzoguanamine resins, cyanamide resins, or aniline resins.

In a particularly preferred embodiment, the use of crosslinkers (III) is completely omitted.

Thickeners (IV) within the meaning of the invention are compounds which make it possible to set the viscosity of the components or their mixtures in such a manner that production and processing of the foam according to the invention is promoted. Suitable thickeners are commercially conventional thickeners such as, for example, natural organic thickeners, for example dextrins or starch, organically modified natural substances, for example cellulose ethers or hydroxyethylcellulose, fully organically synthetic thickeners, for example polyacrylic acids, polyvinylpyrrolidones, poly(meth)acrylic compounds or polyurethanes (associative thickeners) and also inorganic thickeners, for example bentonites or silicic acids. Preferably, use is made of fully organically synthetic thickeners. Particularly preferably, use is made of acrylic thickeners which, before addition, if appropriate are further diluted with water. Preferred commercially conventional thickeners are, for example, Mirox® AM (BGB Stockhausen GmbH, Krefeld, Germany), Walocel® MT 6000 PV (Wolff Cellulosics GmbH & Co KG, Walsrode, Germany), Rheolate® 255 (Elementies Specialities, Ghent, Belgium), Collacral® VL (BASF AG, Ludwigshafen, Germany) and Aristoflex® AVL (Clariant GmbH, Sulzbach, Germany).

Aids (V) within the meaning of the invention are, for example, antioxidants and/or light stabilizers and/or other additives such as, for example, emulsifiers, fillers, plasticizers, pigments, silica sols, aluminum, clay, dispersions, flow enhancers or thixotropic agents, etc.

Cosmetic additives (VI) within the meaning of the invention are, for example, flavourings and aroma substances, abrasives, dyes, sweeteners, etc., and also active ingredients such as fluoride compounds, tooth whiteners, etc.

Foam aids (II), crosslinkers (III), thickeners (IV) and aids (V) can each make up to 20% by weight, and cosmetic additives (VI) up to 80% by weight, based on the foamed and dried gum base.

Preferably, in the method according to the invention, use is made of 80 to 99.5% by weight of the synthetic or chemically modified natural polymers or the starting materials necessary for their formation (I), 0 to 10% by weight of the component (II), 0 to 10% by weight of the component (III), 0 to 10% by weight of the component (IV), 0 to 10% by weight of the component (V) and 0.1 to 20% by weight of the component (VI), the sum being based on the non-volatile fractions of components (I) to (VI), and the sum of the individual components (I) to (VI) adding up to 100% by weight.

Particularly preferably, in the method according to the invention, use is made of 80 to 99.5% by weight of the synthetic or chemically modified natural polymers or the starting materials necessary for their formation (I), 0 to 10% by weight of the component (II), 0 to 10% by weight of the component (IV), 0 to 10% by weight of the component (V) and 0.1 to 15% by weight of the component (VI), the sum being based on the non-volatile fractions of components (I) to (VI), and the sum of the individual components (I) to (VI) adding up to 100% by weight.

Very particular preference is given to 80 to 99.5% by weight of the synthetic or chemically modified natural polymers or the starting materials necessary for their formation (I), 0.1 to 10% by weight of the component (II), 0.1 to 10% by weight of the component (IV), 0.1 to 10% by weight of the component (V) and 0.1 to 15% by weight of the component (VI), the sum being based on the non-volatile fractions of components (I) to (VI), and the sum of the individual components (I) to (VI) adding up to 100% by weight.

The foamed composition can be applied in the most varied manner to the most varied surfaces or in moulds. However, preference is given to casting, doctor-knife application, rolling, spreading, injecting or spraying.

For shaping, the mixture to be foamed or mixture already foamed can first be placed on a surface or into a mould before it is further processed.

Whereas the foamed material, before curing, has a preferred foam density of 200 to 800 g/l, particularly preferably 200 to 700 g/l, very particularly preferably 300 to 600 g/l, the density of the resultant gum base according to the invention after drying is preferably 50 to 600 g/l, particularly preferably 100 to 500 g/l.

The gum bases according to the invention, after the drying step, typically have a thickness of 1 mm to 100 mm, 1 mm to 50 mm, preferably 1 mm to 30 mm.

The gum bases according to the invention can, including in a plurality of layers, for example to produce particularly high foam layers, be applied to the most varied substrates, or cast into moulds.

In addition, the foamed compositions according to the invention can also be used in combination with other support materials such as, for example, textile supports, paper, etc., for example via previous application (for example coating).

The gum bases according to the invention possess excellent mechanical properties, in particular a high extensibility with high tensile strength; thus, after the chewing process return to their original shape, have the capacity to clean the chewing surfaces and sides of the teeth, and do not stick to floor coverings.

In a particular advantageous embodiment of the invention, the synthetic polymers used are polyurethanes in the form of aqueous dispersions (I).

Such polyurethane-polyurea dispersions (I) are obtainable in that

A) isocyanate-functional prepolymers are produced from
a1) organic polyisocyanates
a2) polymeric polyols having number-average molecular weights of 400 to 8000 g/mol and OH functionalities of 1.5 to 6,
a3) if appropriate hydroxyfunctional compounds having molecular weights of 62 to 399 g/mol and
a4) if appropriate hydroxyfunctional, ionic or potentially ionic and/or nonionic hydrophilizing agents,
   B) their free NCO groups are then in whole or in part reacted with
b1) aminofunctional compounds having molecular weights of 32 to 400 g/mol and/or
b2) aminofunctional, ionic or potentially ionic hydrophilizing agents
   with chain extension, and the prepolymers are dispersed before, during, or after step B) in water, if appropriate potentially ionic groups present being able to be converted into the ionic form by partial or complete reaction.

Isocyanate-reactive groups are, for example, amino, hydroxyl or thiol groups.

Of such organic polyisocyanates usable in component a1) are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis-(4,4′-iso-cyanatocyclohexyl)methanes or mixtures thereof of any desired isomer content, 1,4-cyclohexylene diisocyanate, 1,4-phenylene diisocyanate, 2,4- and/or 2,6-toluylene diisocyanate, 1,5-naphthylene diisocyanate, 2,2′- and/or 2,4′- and/or 4,4′-diphenylmethane diisocyanate, 1,3- and/or 1,4-bis-(2-isocyanatoprop-2-yl)-benzene (TMXDI), 1,3-bis(isocyanatomethyl)benzene (XDI), (S)-alkyl 2,6-diisocyanatohexanoates, (L)-alkyl 2,6-diisocyanatohexanoates, having branched, cyclic or acyclic alkyl groups having up to 8 carbon atoms.

In addition to the abovementioned polyisocyanates, use can also be made in conjunction, in proportion, of modified diisocyanates having uretione, isocyanurate, urethane, allophanate, biuret, iminooxadiazinedione and/or oxadiazinetrione structure and also unmodified polyisocyanate having more than 2 NCO groups per molecule for example 4-isocyanatomethyl-1,8-octane diisocyanate (nonane triisocyanate) or triphenylmethane-4,4′,4″-triisocyanate.

Preferably, they are polyisocyanates or polyisocyanate mixtures of the abovementioned type having solely aliphatically and/or cycloaliphatically bound isocyanate groups and an average NCO functionality of the mixture of 2 to 4, preferably 2 to 2.6, and particularly preferably 2 to 2.4.

Particularly preferably, in a1), use is made of 1,6-hexamethylene diisocyanate, isophorone diisocyanate, the isomeric bis-(4,4′-isocyanatocyclohexyl)methanes and also mixtures thereof.

Preferably, in a2), use is made of polymeric polyols having number-average molecular weights of 400 to 6000 g/mol, particularly preferably from 600 to 3000 g/mol.

These preferably have OH functionalities of 1.8 to 3, particularly preferably from 1.9 to 2.1.

Such polymeric polyols are the polyester polyols, polyacrylic polyols, polyurethane polyols, polycarbonate polyols, polyether polyols, polyester polyacrylic polyols, polyurethane polyacrylic polyols, polyurethane polyester polyols, polyurethane polyether polyols, polyurethane polycarbonate polyols and polyesterpolycarbonate polyols which are known per se in polyurethane coating technology. They can be used in a2) individually or in any desired mixtures with one another.

Such polyester polyols are the polycondensates known per se of di- and also if appropriate tri- and tetraols and di- and also if appropriate tri- and tetracarboxylic acids or hydroxycarboxylic acids or lactones. Instead of the free polycarboxylic acids, use can also be made of the corresponding polycarboxylic anhydrides or corresponding polycarboxylic esters of lower alcohols for producing the polyesters.

Examples of suitable diols are ethylene glycol, butylene glycol, diethylene glycol, triethylene glycol, polyalkylene glycols such as polyethylene glycol, in addition 1,2-propanediol, 1,3-propanediol, butane-1,3-diol, butane-1,4-diol, hexane-1,6-diol and isomers, neopentyl glycol or hydroxypivalic neopentyl glycol esters, and also hexane-1,6-diol and isomers, neopentyl glycol and hydroxypivalic neopentyl glycol ester. In addition, use can also be made of polyols such as trimethylol propane, glycerol, erythritol, pentaerythritol, trimethylol benzene or trishydroxyethyl isocyanurate.

As dicarboxylic acids, use can be made of phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. As acid source, use can also be made of the corresponding anhydrides.

If the average functionality of the polyol to be esterified is >2, in addition, use can also be made in conjunction of monocarboxylic acids, such as benzoic acid and hexanecarboxylic acid.

Preferred acids are aliphatic or aromatic acids of the abovementioned type. Particular preference is given to adipic acid, isophthalic acid and phthalic acid.

Hydroxycarboxylic acids which can be used as reaction participants in the production of a polyester polyol having terminal hydroxyl groups are, for example, hydroxy-caproic acid, hydroxybutyric acid, hydroxydecanoic acid, hydroxystearic acid and the like. Suitable lactones are caprolactone, butyrolactone and homologues. Preference is given to caprolactone.

Likewise, in a2), use can be made of hydroxyl-containing polycarbonates, preferably polycarbonatediols, having number-average molecular weights Mₙ of 400 to 8000 g/mol, preferably 600 to 3000 g/mol. These are obtainable by reaction of carbonic acid derivatives such as diphenyl carbonate, dimethyl carbonate or phosgene with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxy-methylcyclohexane, 2-methyl-1,3-propanediol, 3-methyl-1,5-pentanediol, 2,2,4-tri-methylpentane-1,3-diol, dipropylene glycol, polypropylene glycols, dibutylene glycol, polybutylene glycols, bisphenol A, tetrabromobisphenol A and lactone-modified diols of the abovementioned type come into consideration. Mixtures of different diols can also be used.

Preferably, the diol component contains 40 to 100% by weight of hexanediol, preference is given to 1,6-hexanediol and/or hexanediol derivatives. Such hexanediol derivatives are based on hexanediol and have, in addition to terminal OH groups, ester or ether groups. Such derivatives are obtainable by reaction of hexanediol with excess caprolactone, or by etherification of hexanediol with itself to give di- or trihexylene glycol.

Instead of, or in addition to, pure polycarbonatediols, use can also be made in a2) of polyether-polycarbonatediols which contain, as diol component, in addition to the diols described, also polyetherdiols.

Hydroxyl-containing polycarbonates here are preferably of linear structure, but can also contain branched points owing to the incorporation of the polyfunctional components, in particular low-molecular-weight polyols. Suitable substances for this are, for example, glycerol, trimethylol propane, hexane-1,2,6-triol, butane-1,2,4-triol, trimethylol propane, trimethylolethane, pentaerythritol, quinite, mannitol, sorbitol, methyl glycoside or 1,3,4,6-dianhydrohexite.

Suitable polyetherpolyols are, for example, the polytetramethylene glycol polyethers known per se in polyurethane chemistry, as are obtainable by polymerization of tetrahydrofuran by means of cationic ring opening.

Likewise suitable polyetherpolyols are the addition products known per se of styrene oxide, ethylene oxide, propylene oxide, butylene oxides and/or epichlorohydrin to di- or polyfunctional starter molecules.

Suitable starter molecules which can be used are all compounds known from the prior art, as, for example, water, butyl diglycol, glycerol, diethylene glycol, trimethylol propane, propylene glycol, sorbitol, ethylenediamine, triethanolamine, 1,4-butanediol.

Particularly preferred embodiments of the polyurethane dispersions (I) contain, as component a2), a mixture of polycarbonate polyols and polytetramethylene glycol polyols. The fraction of polycarbonate polyols in the mixture is 20 to 80% by weight, and 80 to 20% by weight of polytetramethylene glycol polyols. Preference is given to a fraction of 30 to 75% by weight of polytetramethylene glycol polyols and 25 to 70% by weight of polycarbonate polyols. Particular preference is given to a fraction of 35 to 70% by weight of polytetramethylene glycol polyols and 30 to 65% by weight of polycarbonate polyols, in each case with the proviso that the sum of the percentages by weight of polycarbonate and polytetramethylene glycol polyols gives 100% by weight, and the fraction of the sum of polycarbonate and polytetramethylene glycol polyether polyols in the component a2) is at least 50% by weight, preferably 60% by weight, and particularly preferably at least 70% by weight.

In a3), use can be made of polyols of the said molecular weight range having up to 20 carbon atoms, such as ethylene glycol, diethylene glycol, triethylene glycol, 1,2-propanediol, 1,3-propanediol, 1,4-butanediol, 1,3-butylene glycol, cyclohexane-diol, 1,4-cyclohexanedimethanol, 1,6-hexanediol, neopentyl glycol, hydroquinone dihydroxy ethyl ether, bisphenol A (2,2-bis(4-hydroxyphenyl)propane, hydrogenated bisphenol A, (2,2-bis(4-hydroxycyclohexyl)propane), trimethylol propane, glycerol, pentaerythritol and also any desired mixtures thereof among one another.

Suitable compounds are also ester diols of the said molecular weight range such as α-hydroxybutyl ε-hydroxycaproate, ω-hydroxyhexyl γ-hydroxybutyrate, β-hydroxy-ethyl adipate or bis(β-hydroxyethyl)terephthalate.

In addition, in a3), use can also be made of monofunctional hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Hydroxyfunctional ionic or potentially ionic hydrophilizing agents a4) are taken to mean all compounds which have at least one isocyanate-reactive hydroxyl group and also at least one functionality such as, for example, —COOY, —SO₃Y, —PO(OY)₂(Y⁺ for example ═H⁺, NH₄⁺, metal cation), —NR₂, —NR₃⁺ (R═H, alkyl, aryl), which, on interaction with aqueous media, enter into a pH-dependent dissociation equilibrium and in this manner can be negatively, positively or neutrally charged.

Suitable ionically or potentially ionically hydrophilizing compounds corresponding to the definition of component a4) are, for example, mono- and dihydroxycarboxylic acids, mono- and dihydroxysulphonic acids, and also mono- and dihydroxy-phosphonic acids and salts thereof such as dimethylol propionic acid, dimethylol butyric acid, hydroxypivalic acid, malic acid, citric acid, glycolic acid, lactic acid, the propoxylated adduct of 2-butenediol and NaHSO₃, described, for example in DE-A 2 446 440 (pages 5-9, formulae I-III) and also compounds which contain, as hydrophilic structural components, for example amine-based building blocks such as N-methyldiethanolamine convertible into cationic groups.

Preferred ionic or potentially ionic hydrophilizing agents of the component a4) are those of the abovementioned type which act in a hydrophilizing manner anionically, preferably via carboxyl or carboxylate and/or sulphonate groups.

Particularly preferred ionic or potentially ionic hydrophilizing agents are those which contain carboxyl and/or sulphonate groups as anionic or potentially anionic groups such as the salts of dimethylol propionic acid or dimethylol butyric acid.

Suitable nonionically hydrophilizing compounds of the component a4) are, for example, polyoxyalkylene ethers which contain at least one hydroxyl or amino group as isocyanate-reactive group.

Examples are the monohydroxy functional polyalkylene oxide polyether alcohols having a statistical mean of 5 to 70, preferably 7 to 55, ethylene oxide units per molecule, such as are accessible in a manner known per se by alkoxylating suitable starter molecules (e.g. in Ullmanns Encyclopadie der technischen Chemie [Ullmann's Encyclopaedia of Industrial Chemistry], 4th edition, volume 19, Verlag Chemie, Weinheim, pages 31-38).

These are either pure polyethylene oxide ethers or mixed polyalkylene oxide ethers, with them containing at least 30 mol %, preferably at least 40 mol %, ethylene oxide units, based on all alkylene oxide units present.

Particularly preferred nonionic compounds are monofunctional mixed polyalkylene oxide polyethers which have 40 to 100 mol % ethylene oxide and 0 to 60 mol % propylene oxide units.

Suitable starter molecules for such nonionic hydrophilizing agents are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane, or tetrahydrofurfuryl alcohol, diethylene glycol monoalkyl ethers, such as, for example, diethylene glycol monobutyl ether, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine and also heterocyclic secondary amines such as morpholine, pyrrolidine, piperidine or 1H-pyrazole. Preferred starter molecules are saturated monoalcohols of the abovementioned type. Particularly preferably, as starter molecules, use is made of diethylene glycol monobutyl ether, or n-butanol.

Alkylene oxides suitable for the alkoxylation reaction are, in particular, ethylene oxide and propylene oxide, which can be used in the alkoxylation reaction in any desired sequence or else in a mixture.

As component b1), use can be made of di- or polyamines such as 1,2-ethylenediamine, 1,2- and 1,3-diaminopropane, 1,4-diaminobutane, 1,6-diamino-hexane, isophoronediamine, mixtures of isomers of 2,2,4- and 2,4,4-trimethyl-hexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4-diaminodicyclohexylmethane and/or dimethylethylenediamine. The use of hydrazine or also hydrazides such as adipic dihydrazide is likewise possible.

In addition, as component b1), use can also be made of compounds which, in addition to a primary amino group, also have secondary amino groups or, in addition to an amino group (primary or secondary), also have OH groups. Examples of these are primary/secondary amines such as diethanolamine, 3-amino-1-methyl-aminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

In addition, as component b1), use can also be made of monofunctional amine compounds such as, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof, amidoamines of diprimary amines and monocarboxylic acids, monoketimines of diprimary amines, primary/tertiary amines such as N,N-dimethylaminopropylamine.

Preferably, use is made of 1,2-ethylenediamine, hydrazine hydrate, 1,4-diamino-butane, isophoronediamine and diethylenetriamine.

Ionically or potentially ionically hydrophilizing compounds of the component b2) are taken to mean all compounds which have at least one isocyanate-reactive amino group and also at least one functionality such as, for example, —COOY, —SO₃Y, —PO(OY)₂ (Y for example=H, NH₄⁺, metal cation), —NR₂, —NR₃⁺ (R═H, alkyl, aryl), which, on interaction with aqueous media, enter into a pH-dependent dissociation equilibrium and in this manner can be positively, negatively or neutrally charged.

Suitable ionically or potentially ionically hydrophilizing compounds are, for example, mono- and diaminocarboxylic acids, mono-1- and diaminosulphonic acids and also mono- and diaminophosphonic acids and salts thereof. Examples of such ionic or potentially ionic hydrophilizing agents are N-(2-aminoethyl)-β-alanine, 2-(2-aminoethylamino)ethanesulphonic acid, ethylenediaminepropylsulphonic or butylsulphonic acid, 1,2- or 1,3-propylenediamine-β-ethylsulphonic acid, glycine, alanine, taurine, lysine, 3,5-diaminobenzoic acid and the addition product of IPDI and acrylic acid (EP-A 0 916 647, Example 1). In addition, use can be made of cyclohexylaminopropanesulphonic acid (CAPS) from WO-A 01/88006 as anionic or potentially anionic hydrophilizing agent.

Preferred ionic or potentially ionic hydrophilizing agents of the component b2) are those of the abovementioned type which act in a hydrophilizing manner via anionic, preferably carboxyl groups or carboxylate groups and/or sulphonate groups.

Particularly preferred ionic or potentially ionic hydrophilizing agents b2) are those which contain carboxyl and/or sulphonate groups as anionic or potentially anionic groups, such as the salts of N-(2-aminoethyl)-β-alanine, 2-(2-aminoethylamino)ethanesulphonic acid or the addition product of IPDI and acrylic acid (EP-A 0 916 647, Example 1).

For the hydrophilization, preferably use is made of a mixture of anionic or potentially anionic hydrophilizing agents and nonionic hydrophilizing agents.

The ratio of NCO groups of the compounds of component a1) to NCO-reactive groups of the components a2) to a4) in the production of the NCO-functional prepolymer is 1.05 to 3.5, preferably 1.2 to 3.0, particularly preferably 1.3 to 2.5.

The aminofunctional compounds in stage B) are used in an amount such that the equivalent ratio of isocyanate-reactive amino groups of these compounds to the free isocyanate groups of the prepolymer is 40 to 150%, preferably 50 to 125%, particularly preferably 60 to 120%.

In a preferred embodiment, use is made of anionically and nonionically hydrophilized polyurethane dispersions, for their production use being made of the components a1) to a4) and b1) to b2) in the following amounts, the individual amounts totaling 100% by weight:

5 to 40% by weight of component a1),
55 to 90% by weight of a2),
0.5 to 20% by weight sum of components a3) and b1)
0.1 to 25% by weight sum of components a4) and b2), based on the total amounts of components a1) to a4) and b1) to b2), use being made of 0.1 to 5% by weight of anionic or potentially anionic hydrophilizing agents a4) and b2).

Particularly preferably, the amounts of components a1) to a4) and b1) and b2) are as follows:

5 to 35% by weight of component a1),
60 to 90% by weight of a2),
0.5 to 15% by weight sum of components a3) and b1)
0.1 to 15% by weight sum of components a4) and b2), based on the total amounts of components a1) to a4) and b1) to b2), use being made of 0.2 to 4% by weight of anionic or potentially anionic hydrophilizing agents a4) and b2).

Very particularly preferably, the amounts of components a1) to a4) and b1) and b2) are as follows:

10 to 30% by weight of component a1),
65 to 85% by weight of a2),
0.5 to 14% by weight sum of components a3) and b1)
0.1 to 13.5% by weight sum of components a4) and b2), based on the total amounts of components a1) to a4), use being made of 0.5 to 3.0% by weight of anionic or potentially anionic hydrophilizing agents.

Particularly preferred embodiments of the polyurethane dispersions (I), as component a1), contain isophorone diisocyanate and/or 1,6-hexamethylene diisocyanate and/or the isomeric bis(4,4′-isocyanatocyclohexyl)methanes in combination with a2) of a mixture of polycarbonate polyols and polytetramethylene glycol polyols.

The fraction of polycarbonate polyols in the mixture a2) is 20 to 80% by weight, and 80 to 20% by weight of polytetramethylene glycol polyols. Preference is given to a fraction of 30 to 75% by weight of polytetramethylene glycol polyols and 25 to 70% by weight of polycarbonate polyols. Particular preference is given to a fraction of 35 to 70% by weight of polytetramethylene glycol polyols and 30 to 65% by weight of polycarbonate polyols, in each case with the proviso that the sum of the percentages by weight of the polycarbonate and polytetramethylene glycol polyols gives 100% by weight and the fraction of the sum of polycarbonate and polytetramethylene glycol polyether polyols of the component a2) is at least 50% by weight, preferably 60% by weight, and particularly preferably at least 70% by weight.

Such polyurethane dispersions can be produced in one or more stage(s) in homogeneous or multistage reaction, partially in disperse phase. After polyaddition, complete or carried out in part, of a1) to a4), a dispersion, emulsification or solution step proceeds. Subsequently, if appropriate, further polyaddition or modification in disperse phase proceeds.

All methods known from the prior art can be used here such as, for example, prepolymer mixing methods, acetone methods or melt dispersion methods. Preferably, the process proceeds via the acetone method.

For preparation according to the acetone method, customarily components a2) to a4) which must not have any primary or secondary amino groups, and the polyisocyanate component a1), for production of an isocyanate-functional polyurethane prepolymer, are charged in whole or in part and if appropriate diluted with a solvent which is water-miscible but inert to isocyanate groups, and heated to temperatures in the range from 50 to 120° C. To accelerate the isocyanate addition reaction, the catalysts known in polyurethane chemistry can be added.

Suitable solvents are the customary aliphatic, ketofunctional solvents such as acetone, 2-butanone, which can be added not only at the start of production, but also, if appropriate, in parts later. Preference is given to acetone and 2-butanone.

Other solvents (cosolvents) such as xylene, toluene, cyclohexane, butyl acetate, methoxypropyl acetate, N-methylpyrrolidone, N-ethylpyrrolidone, solvents having ether or ester units can additionally be used and completely or in part distilled off or, in the case of N-methylpyrrolidone, N-ethylpyrrolidone, remain completely in the dispersion.

In a particular embodiment of the invention, the use of cosolvents is avoided completely.

Subsequently any components of a1) to a4) which are not yet added at the start of the reaction are added.

The reaction of components a1) to a4) to form the prepolymer proceeds partially or completely, but preferably completely. In such a manner polyurethane prepolymers which contain free isocyanate groups are obtained in the absence of solvent or in solution.

In the neutralization step for the partial or complete conversion of potentially anionic groups to anionic groups, use is made of bases such as tertiary amines, for example trialkylamines having 1 to 12, preferably 1 to 6, carbon atoms in each alkyl radical, or alkali metal bases such as the corresponding hydroxides.

Examples of these are trimethylamine, triethylamine, methyldiethylamine, tripropyl-amine, N-methylmorpholine, methyldiisopropyl amine, ethyldiisopropylamine and diisopropylethylamine. The alkyl radicals can also bear, for example hydroxyl groups, such as in dialkylmonoalkanolamines, alkyldialkanolamines and trialkanolamines. As neutralizing agents, if appropriate, use can also be made of inorganic bases such as aqueous ammonia solution or sodium hydroxide or potassium hydroxide.

Preference is given to ammonia, triethylamine, triethanolamine, dimethylethanol-amine or diisopropylethylamine and also sodium hydroxide.

In the case of cationic groups, use is made of dimethyl sulphate or succinic acid or phosphoric acid.

The amount of the bases is 50 and 125 mol %, preferably between 70 and 100 mol % of the amount of substance of the acid groups to be neutralized. The neutralization can also proceed simultaneously with dispersion by the dispersion water already containing the neutralising agent.

Subsequently, in a further method step, if this has not yet proceeded, or only in part, the resultant prepolymer is dissolved using aliphatic ketones such as acetone or 2-butanone.

The amine components b1), b2) can if appropriate be used individually or in mixtures in water- or solvent-diluted form in the method according to the invention, in principle any sequence of addition being possible.

If water or organic solvents are used in conjunction as diluents, the diluent content in the component used in b) for chain extension is preferably 70 to 95% by weight.

Dispersion preferably proceeds subsequent to chain extension. For this the dissolved and chain-lengthened polyurethane polymer, if appropriate under severe shear, for example vigorous stirring, is either charged into the dispersion water, or, vice versa, the dispersion water is stirred into the chain-lengthened polyurethane polymer solutions. Preferably, the water is added to the dissolved chain-lengthened polyurethane polymer.

The solvent still present in the dispersions after the dispersion step is customarily subsequently removed by distillation. It is also possible for removal to proceed even during dispersion.

The residual content of organic solvents in the dispersions essential to the invention is typically less than 1.0% by weight, preferably less than 0.5% by weight, particularly preferably less than 0.1% by weight, very particularly preferably less than 0.05% by weight, based on the total dispersion.

The pH of the dispersions essential to the invention is typically less than 9.0, preferably less than 8.5, particularly preferably less than 8.0.

The solids content of the polyurethane dispersion is typically 20 to 70% by weight, preferably 30 to 65% by weight, particularly preferably 40 to 63% by weight, and very particularly preferably 50 to 63% by weight.

In addition, it is possible to modify the polyurethane-polyurea dispersions (I) which are essential to the invention by polyacrylates. For this, in the presence of the polyurethane dispersion, an emulsion polymerization of olefinically unsaturated monomers, for example esters of (meth)acrylic acid and alcohols having 1 to 18 carbon atoms, styrene, vinyl esters or butadiene is carried out, as described, for example, in DE-A-1 953 348, EP-A-0 167 188, EP-A-0 189 945 and EP-A-0 308 115. The monomers contain one or more olefinic double bonds. In addition, the monomers can contain functional groups such as hydroxyl, epoxide, methylol or acetoacetoxy groups.

In a particular preferred embodiment of the invention, this modification is omitted.

In principle it is possible to mix the polyurethane-polyurea dispersions (I) essential to the invention with other aqueous binders. Such aqueous binders can be made up, for example, of polyester, polyacrylic, polyepoxy or polyurethane polymers. The combination of radiation-curable binders, as are described, for example, in EP-A-0 753 531 is also possible. It is likewise possible to blend the polyurethane-polyurea dispersions (I) with other anionic or nonionic dispersions such as, for example, polyvinyl acetate, polyethylene, polystyrene, polybutadiene, polyvinyl chloride, polyacrylates and copolymer dispersions.

In a particularly preferred embodiment of the invention, this modification is omitted.

### EXAMPLES

Unless stated otherwise, all percentages relate to the weight.

The solid contents were determined as specified in DIN-EN ISO 3251.

NCO contents were determined, unless explicitly stated otherwise, volumetrically as specified in DIN-EN ISO 11909.

### Substances and Abbreviations Used:

Diaminosulphonate: NH₂—CH₂CH₂—NH—CH₂CH₂—SO₃Na (45% strength in water)
Desmophen® C2200: Polycarbonate polyol, OH number 56 mg of KOH/g, number-average molecular weight 2000 g/mol (Bayer Materialscience AG, Leverkusen, DE)
PolyTHF® 2000: Polytetramethylene glycol polyol, OH number 56 mg of KOH/g, number-average molecular weight 2000 g/mol (Basf Ag, Ludwigshafen, DE)
PolyTHF® 1000: Polytetramethylene glycol polyol, OH number 112 mg of KOH/g, number-average molecular weight 1000 g/mol (Basf Ag, Ludwigshafen, DE)
Polyether LB 25: (Monofunctional polyether based on ethylene oxide/propylene oxide, number-average molecular weight 2250 g/mol, OH number 25 mg of KOH/g (Bayer Materialscience AG, Leverkusen, DE)
Stokal® STA: Foam aid based on ammonium stearate, active ingredient content: 30% (Bozzetto GmbH, Krefeld, DE)
Stokal® SR: Foam aid based on succinamate, active ingredient content: approximately 34% (Bozzetto GmbH, Krefeld, DE)
Mirox AM: Aqueous acrylic acid copolymer dispersion (BGB Stockhausen GmbH, Krefeld, DE)
Borchigel ALA: Aqueous, anionic acrylic polymer solution (Borchers GmbH, Langenfeld, DE)
Octosol SLS: Aqueous sodium lauryl sulphate solution (Tiarco Chemical Europe GmbH, Nuremberg, DE)
Octosol 845 Sodium lauryl sulphate ether (Tiarco Chemical Europe GmbH, Nuremberg, DE)

The average particle sizes (the number average is given) of the PUR dispersions were determined by means of laser correlation spectroscopy (device: Malvern Zetasizer 1000, Malver Inst. Limited).

### Example 1

### PUR Dispersion (Component I)

144.5 g of Desmophen® C2200, 188.3 g of PolyTHF® 2000, 71.3 g of PolyTHF® 1000 and 13.5 g of Polyether LB 25 were heated to 70° C. Subsequently, at 70° C., in the course of 5 min, a mixture of 45.2 g of hexamethylene diisocyanate and 59.8 g of isophorone diisocyanate was added and the mixture was stirred under reflux until the theoretical NCO value was achieved. The finished prepolymer was dissolved with 1040 g of acetone at 50° C. and subsequently a solution of 1.8 g of hydrazine hydrate, 9.18 g of diaminosulphonate and 41.9 g of water was added in the course of 10 min. The post-stirring time was 10 min. After addition of a solution of 21.3 g of isophoronediamine and 106.8 g of water, the mixture was dispersed in the course of 10 min by addition of 254 g of water. Removal of the solvent by distillation in vacuo followed, and a storage-stable dispersion having a solids content of 60.0% was obtained.

### Example 2

### PUR Dispersion (Component I)

2159.6 g of a difunctional polyester polyol based on adipic acid, neopentyl glycol and hexanediol (mean molecular weight 1700 g/mol, OH number=66), 72.9 g of a monofunctional polyether based on ethylene oxide/propylene oxide (70/30) (mean molecular weight 2250 g/mol, OH number 25 mg of KOH/g) were heated to 65° C. Subsequently, at 65° C., in the course of 5 min, a mixture of 241.8 g of hexamethylene diisocyanate and 320.1 g of isophorone diisocyanate was added and stirred at 100° C. until the theoretical NCO value of 4.79% was achieved. The finished prepolymer was dissolved with 4990 g of acetone at 50° C. and subsequently a solution of 187.1 g of isophoronediamine and 322.7 g of acetone was added in the course of 2 min. The post-stirring time was 5 min. Subsequently, in the course of 5 min, a solution of 63.6 g of diaminosulphonate, 6.5 g of hydrazine hydrate and 331.7 g of water was added. The mixture was dispersed by adding 1640.4 g of water. The solvent was then removed by distillation in vacuo and a storage-stable PUR dispersion having a solids content of 58.9% was obtained.

### Example 3

### PUR Dispersion (Component 1)

2210.0 g of a difunctional polyester polyol based on adipic acid, neopentyl glycol and hexanediol (mean molecular weight 1700 g/mol, OH number=66) were heated to 65° C. Subsequently, at 65° C., in the course of 5 min, a mixture of 195.5 g of hexamethylene diisocyanate and 258.3 g of isophorone diisocyanate was added and stirred at 100° C. until the theoretical NCO value of 3.24% was reached. The finished prepolymer was dissolved with 4800 g of acetone at 50° C. and subsequently a solution of 29.7 g of ethylenediamine, 95.7 g of diaminosulphonate and 602 g of water was added in the course of 5 min. The post-stirring time was 15 min. Subsequently, in the course of 20 min, the mixture was dispersed by adding 1169 g of water. The solvent was then removed by distillation in vacuo and a storage-stable PUR dispersion having a solids content of 60% was obtained.

### Example 4

### Production of a Gum Base According to the Invention

1000 g of a commercially available polyurethane dispersion (I) (Impranil DLU, Bayer MaterialScience AG, Germany) were mixed with 15 g of Stokal STA (II), 20 g of Stokal SR (II) and 30 g of Borchigel ALA (IV) and subsequently foamed by introducing air using a hand mixing apparatus. The resultant foam density was 400 g/l. Thereafter the foamed paste was applied using a film-drawing apparatus consisting of two polished rolls which could be set to an exact distance, and in front of the rear roll, a separation paper was inserted. Using a feeler gauge, the distance between paper and front roll was set. This distance corresponded to the film thickness (wet) of the resultant coating which was selected in such a manner that a dry layer thickness >100 μm was achieved. Subsequently, the material was dried in a drying cabinet at 80° C. for 15 minutes. After taking off the separation paper, the gum base according to the invention was obtained. The performance properties are shown in Table 1.

### Example 5

### Production of a Gum Base According to the Invention

1000 g of the dispersion (I) obtained from Example 1 were mixed with 30 g of Octosol SLS (II), 20 g of Stokal SR (II), 20 g of Octosol 845 (II), 5 g of 5% strength ammonia solution and 15 g of Mirox AM (IV) and subsequently foamed by introducing air using a hand mixing apparatus. The resultant foam density was 400 g/l. Thereafter, the foamed paste was applied using a film-drawing apparatus consisting of two polished rolls which could be set to an exact distance, and in front of the rear roll a separation paper being inserted. Using a feeler gauge, the distance between paper and front roll was set. This distance corresponded to the film thickness (wet) of the resultant coating which was selected in such a manner that a dry layer thickness >100 μm was achieved. Subsequently, the material was dried in a drying cabinet at 80° C. for 15 minutes. After taking off the separation paper, the gum base according to the invention was obtained. The performance properties are shown in Table 1.

TABLE 1
Performance properties of the gum bases
according to the invention

| | 100% modulus | Extension | Tensile strength |
|---|---|---|---|
| Gum base from: | [MPa] | [%] | [MPa] |
| Example 4 | 0.6 | 570 | 2.4 |
| Example 5 | 0.8 | 710 | 5.2 |

The modulus at 100% extension was determined on films having a layer thickness >100 μm.

| Gum base from:. | σ_{f}/E | R × E/σ_{f}² |
|---|---|---|
| Example 4 | 1.4 | 1.5 |
| Example 5 | 1.6 | 1.3 |

σ_{f}: tensile strength
E: modulus of elasticity
R: resistance to tear

1-9. (canceled)

10. Gum bases made from foamed synthetic or natural chemically modified polymers.

11. The gum bases according to claim 10, wherein they are not thermoplastic.

12. The gum bases according to claim 10, wherein they have a tensile modulus at 100% extension of 0.3 to 3.5 MPa, at a tensile strength of 0.5 to 40 MPa and an extensibility of 200 to 2000%.

13. The gum bases according to claim 10, wherein these have a ratio of tensile strength to modulus of elasticity of greater than or equal to 1 and a ratio of the product of resistance to tear (as specified in DIN ISO 34-1 (2004)) and modulus of elasticity to the square of the tensile strength less than 4 mm.

14. A method for producing gum bases according to claim 10 in which synthetic or chemically modified natural polymers or the starting materials necessary for their formation (I), optionally together with further components of the gum bases, are foamed and simultaneously or subsequently cured to obtain the foam structure.

15. The method according to claim 14, wherein the foamed material, before curing, has a foam density of 200 to 800 g/1.

16. The method according to claim 14, wherein the gum base obtained after the curing has a foam density of 50 to 600 g/l.

17. The method according to claim 15, wherein the synthetic polymers are optionally thermoplastic polyurethanes, polyvinyl chloride plastisols, low-density polyethylene (LDPE), ethylene-vinyl acetate copolymers (EVA), synthetic or natural rubber or silicone rubber.

18. The method according to claim 14, wherein, in addition to the synthetic or chemically modified natural polymers or the starting 5 materials necessary for their formation (I), use is also made in conjunction of foam aids (II), crosslinkers (III), thickeners (IV), aids (V) and/or cosmetic additives (VI).
